(19) United States
(12) Patent Application Publication
(10) Pub. No.: US 2002/0090631 A1
(43) Pub. Date: Jul. 11, 2002

(54) Method for predicting protein binding from primary structure data

(76) Inventors: David A. Gough, Cardiff, CA (US); Joel R. Bock, El Cajon, CA (US)

Correspondence Address: Colleen J. McKiernan, Brown Martin Haller & McClain, 1660 Union Street, San Diego, CA, 92101, US

(21) Appl. No.: 09/993,272

(22) Filed: Nov. 14, 2001

Related U.S. Application Data
(60) Provisional application No. 60/248,258, filed on Nov. 14, 2000.

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

The invention is a teachable system and method for predicting the interactions of proteins with other proteins, nucleic acids and small molecules. A database containing protein sequences and information regarding protein interactions is used to “teach” the machine. Proteins with unknown interactions are compared by the machine to proteins in the database. Homologs of proteins known to interact in the database are predicted to interact.

### CROSS-REFERENCES TO RELATED APPLICATIONS

This application claims the benefit of priority of U.S. provisional application Serial No. 60/248,258 filed Nov. 14, 2001 which is incorporated herein by reference in its entirety.

### COMPUTER APPENDIX

A computer program listing appendix submitted in duplicate on compact disc under § 1.52 ((e) 5) with the application is hereby incorporated by reference.

### FIELD OF THE INVENTION

The invention is a trainable system and computational method for predicting the interaction of biopolymers with other biopolymers, nucleic acids, and with a variety of ligands based on the sequence or primary structure of the biomolecule.

### BACKGROUND OF THE INVENTION

Determination of protein-protein interaction is a slow and cumbersome process. Methods such as the yeast two-hybrid system can reveal unexpected, transient protein-protein interactions in cells. Alternatively, more stable protein-protein interactions may be determined by immunoprecipitations and other in vitro binding assays. However, it is generally not possible to determine the specific sites of interaction between the proteins by these methods. High-resolution structural analysis can reveal protein-protein interactions at a molecular level. Structures can be obtained for protein complexes, but only proteins already known to interact would be studied in this manner. Pairs of proteins may be studied individually to predict protein-protein interactions, but there is no high-throughput method to search for proteins that will likely interact with a protein of interest. Even if such a method did exist, it would be limited by the number of protein structures that are available in databases.

Similarly, methods to determine protein-nucleic acid interactions and protein-ligand binding interactions are also cumbersome. A number of binding assays, both in vitro and in vivo have been developed depending on the interaction to be analyzed. Although some of these methods may be relatively high throughput, based on 96-well plates with automated read out, the process of analyzing 10,000 compounds produced by combinatorial chemistry can be daunting.

Computational prediction of interactions has involved estimation of the site of interaction, utilization of features and properties related to interface topology, solvent accessible surface area, and hydrophobicity, or the recognition of specific residue or geometric motifs. These computational methods are highly specialized, require specific physiochemical information that is generally not available for all proteins, and are not broadly applicable.

Genome projects in a variety of organisms have provided researchers with a large amount of DNA sequence information. Gene chip technology has provided a means to analyze gene expression under a variety of conditions, including development and disease. However, although genes can frequently be assigned into groups based on DNA sequence (e.g. kinases, transcription factors, structural proteins, etc), the way that the proteins interact is not revealed by DNA sequence.

Protein function is exceedingly diverse. Within the cell, proteins assemble into complex and dynamic macromolecular structures, recognize and degrade foreign molecules, regulate metabolic pathways, control DNA replication and progression through the cell cycle, synthesize other chemical species, facilitate molecular recognition, localize and “scaffold” other proteins within signal transduction cascades and participate in other important functions.

To appreciate the breadth of protein function, a description of protein-protein interactions is a necessary first step. Beginning with the proteomic constituents, a rational research strategy should then proceed in the direction of abstract information flow represented by interaction→network→function rather than the more typical function→interaction→network.

Given the volume of proteomic data generated by high-throughput technologies, prediction of protein function requires integration of empirical data with bioinformatic comparative prediction analyses. For example, a complete pairwise protein interaction in the relatively tiny proteome of the bacterium Mycoplasma genitalium, with N=486 proteins, requires screening of N(N−1) or 235,710 separate interactions (EBI Proteome Analysis database; http://www.ebi.ac.uk/proteome). The task would be overwhelming if approached by experiment alone.

The workhorse of experimental proteomics has been the two hybrid screen (Fields and Song, 1989), which has been criticized based on the accuracy of the results and its labor intensive nature (Enright et al., 1999). Protein chips may eventually provide large scale simultaneous protein-protein interaction data (MacBeath and Schreiber, 2000), but technical problems (denaturing, substrate biocompatablity) must be overcome to scale-up for high-throughput analysis. Moreover, the preparation of chips is non-trivial. As application of proteins from cell or tissue homogenates directly to the chip would not be possible as the resulting chip would be coated with predominantly structural proteins which tend to represent the plurality of cell proteins. Unlike nucleic acids that may be amplified from a chip, the small amounts of protein on a chip would be insufficient for sequencing. Therefore, proteins would need to be expressed and applied to a chip at distinct locations to allow for identification of the protein bound by the probe. An individual chip would need to be prepared for the analysis of every few protein probes depending on multiplex capacity of the system. Improved technologies are required before protein chip technology is practical and affordable.

Other approaches may become prominent as proteomics technology continues to evolve: for example, denaturing may be avoided by combining high performance liquid chromatography (HPLC) co-elution with MALDI-TOF (Matrix Assisted Laser Desorption Ionization) mass spectrometry (Champion et al, 2001). Thus, one may isolate complexes by chromatography, separate the components of the complex and identify them by sequencing then individually. Such systems do not allow for the definition of individual protein-protein interactions, but instead provide information on complexes which then must be analyzed by further experimentation to determine the individual interactions.

### SUMMARY OF THE INVENTION

The invention is a trainable system and method for the prediction of the interactions, mutual bindings or associations between specific homogenous pairings of biomolecules such as, but not limited to, protein-protein, DNA-DNA, and heterogenous pairings such as protein-DNA, protein-RNA, DNA-RNA, etc. The predictions are based on primary protein sequence available in electronic format and associated physiochemical information also available in electronic format such as hydrophobicity, charge and chemical composition.

For example, primary structure of a vast number of proteins is now available in electronic format, with associated physiochemical properties of each amino acid. These data can be digitally encoded as a sequence of numbers, this new sequence representing the properties of each protein in potential binding interaction. The trainable system is trained to recognize patterns in these sequences, specifically patterns that characterize positive interaction with between proteins as observed experimentally. This system makes a statistical decision as to whether or not a new pair of proteins will interact, based on its “training” from previous data. The system achieves a high degree of precision relative to previous methods in making these decisions, enabling higher throughput screening of potential candidate proteins for different applications.

The invention can be applied to larger scale studies of protein-protein interactions in a proteome wide scale. Application of a “phylogenetic bootstrap” method for protein-protein interaction mining, which comprises traversal of a phenogram, interleaving rounds of computation and experiment, to develop a knowledge base of protein interactions in genetically similar organisms. The steps comprising phylogenetic bootstrap are distilled into an algorithm, described herein in detail. Similar methods can be applied to predict interactions of other types of biomolecules.

### BRIEF DESCRIPTION OF THE DRAWINGS

The present invention will be better understood from the following detailed description of an exemplary embodiment of the invention, taken in conjunction with the accompanying drawings in which like reference numerals refer to like parts and in which:

FIG. 1. Scatterplot showing detail view of sample datapoints xᵢε [symbol] ⁿ representing H. pylori protien-protein interactions, visualized by two dimensional Sammon mapping. Circled points indicate incorrect decisions made during leave-one-out prediction error estimation. 90% of all data points (1,873/2,077) appear in this map. Coordinate axes contain arbitrary units. Estimated system generalization error rate is 12.04%.

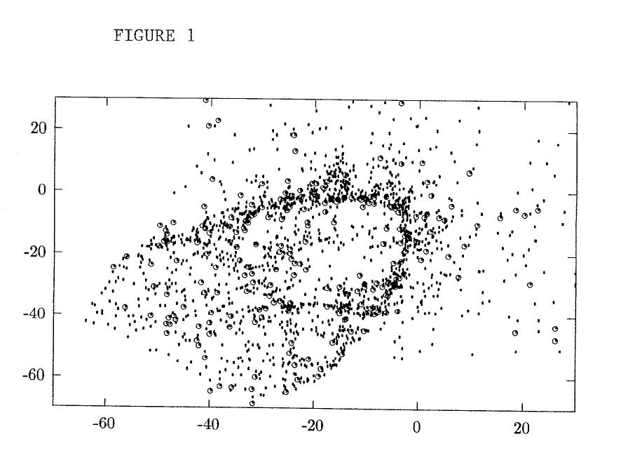

### DETAILED DESCRIPTION AND PREFERRED EMBODIMENTS

The invention is a method of representing biopolymers in a computational trainable system for use in the prediction of the interaction of proteins with other proteins, nucleic acids, small molecules and biopolymers. The interactions are determined in a pairwise fashion, with higher order structures containing more than two components being determined in multiple rounds of analysis. A collection of known biomolecular interactions, such as protein-protein interactions, are encoded as a set of features on a residue-by-residue basis in the trainable system. Databases of heterogenous protein-protein interactions exist, including the publicly-accessible Database of Interacting Proteins (DIP: http://dip.doe-mbi.ucla.edu) which at the time of this application contains 10933 interaction pairs. Other databases contain information regarding protein interactions in single organisms; one such database is available at http://pim.hybrigenics.com,which contains all of the known protein-protein interactions known in the bacteria H. pylori. The selection of a database is not a limiting aspect of the invention. Moreover, the databases listed should not be considered static entities or to be limited to the data that they contain at the time of the application. The databases are a source of training sets to “teach” the trainable system, but are not a component of the invention itself. The invention is instead the manner in which the biopolymers are represented as a linear set of features and used in the trainable system to predict the interactions of the encoded biopolymers with other molecules.

The accuracy of the predictive model is dependent upon the quality of the database used. The more the system is “taught” in the number of biomolecular interactions entered into the database, and the greater the similarities between the molecules to be compared, the higher the predictive value of the model will be. Alternatively, limiting the members of the query group to a single cell compartment (e.g. endoplasmic reticulum, nucleus, Golgi apparatus) increases the accuracy of the predictive model by eliminating possible interactions between proteins that would never come into contact with each other in the context of the cell.

A trainable system is defined as a program, algorithm or other analytical method into which data are input in the form of a training set from which the system can “learn” to determine patterns and that will allow for predictions of outcomes, upon analysis of unknowns similar to those in the training set. “Learning” and analysis of the unknown samples may be performed by any of a number of methods including the use of a support vector machine (SVM), neural network, classification and regression analysis (CART), Bayesian networks, or other algorithms, software programs or a combination thereof. In the instant invention the training set is a group of pairs of biomolecules that do or do not interact that are used to “teach” the system what characteristic features do or do not interact such that the unknowns can be analyzed for the presence of features such that interactions may be predicted. The training set may be augmented or modified and should not be considered a static entity. The invention is not limited by the algorithm, software or hardware used, but instead is dependent on the method used to train the system such that predictions on interactions can be made based on linear sequence information or primary structure of biomolecules, rather than based on tertiary structure.

A training set is defined as a collection of data, typically derived from a database, containing examples of pairs of biomolecules that do or do not interact. The examples of biomolecular interaction or non-interaction are analyzed by a trainable system so it may “learn” how classes of biomolecules interact. The type of biomolecular interactions to be determined (e.g. protein-protein, protein-nucleic acid) in the group of biomolecules with unknown interactions would determine the selection of the type of training set. The training set may be augmented or modified during the process of analysis.

A biomolecule is defined as a protein, peptide, nucleic acid, complex lipid or carbohydrate, small molecule such as a growth factor, hormone, vitamin, lipid, carbohydrate, neurotransmitter, signalling molecule, amino acid or nucleotide, a scaffold for attachment of cells, a polymer for the use in the assembly of organ, joint or other implant, a bioactive agent such as a drug.

Primary or linear structure is defined as the sequence of nucleotides or amino acids in the nucleic acid or polypeptide of interest, respectively. The primary structure of a biomolecule is defined as a representation of orgainc or inorgainc molecules as a sequence of constituient elements.

For example, in the invention, a training set “teaches” the trainable system about biomolecular interactions by providing examples of how proteins interact with each other by providing a number of examples of protein-protein interactions. Proteins in the query group are matched to the proteins in the database based on homology. Proteins in the query group are predicted to interact based on the interactions of their homologs in the database. For example, if protein A in the database is homologous to protein A′ of the query group either in a portion or along the entire length of the protein, and protein B in the database is homologous to protein B′ in the query group, and proteins A and B are known to interact, proteins A′ and B′ are predicted to interact. As interactions tend to take place through modular domains in the protein (e.g. SH2 and SH3 domains, zinc fingers, leucine zippers, amphipathic helicies), predictions may be made accurately even if the proteins in the query group do not have overall high homology to proteins in the database. However, the greater similarity of the organisms in the query and database groups, the better the prediction accuracy of the method.

The invention is a method for whole-proteome interaction mapping wherein, the database comprises all of the experimentally-known or hypothesized protein-protein interactions of a single organism. Protein sequences comprising a partial or complete proteome from a different organism, that may or may not contain any defined protein-protein interaction, are analyzed by the trainable system for homology between proteins in the database and the query group. Homologous proteins of interacting pairs in the database are predicted to interact with each other. Proteins are analyzed on an all-against-all basis with each potential pairwise combination being analyzed. The learning machine may be used for subsequent rounds of analysis to predict higher order structures containing greater than two proteins.

Data obtained through use of the trainable system can be tested in a laboratory setting to confirm interactions. Such data can be entered into the system for subsequent rounds of analysis and to further “teach” the system about additional protein-protein interactions. As more data are entered into the system, the predictive ability of the system increases.

The invention is a method for the use of a trainable system to predict the presence of epitopes of interest, including functional domains and binding sites of proteins, and antigenic determinants. By casting the numerical optimization procedure as a regression problem, a continuous value for binding affinity of ligand-molecular complex can be learned. In this manner the same scheme for representing linear biopolymer sequences as features is used, and the training procedure involves “sliding” a window along the query sequence, each step outputting a numerical value that constitutes a predicted interaction value of the sequence within the window and the query ligand. Example public-domain databases containing data appropriate for training the system in this mode are: (1) The Ligand Chemical Database for Enzyme Reactions (http://www.genome.ad.jp/dbget/ligand.html), (2) The Function Immunology Database of MHC molecules, antigens and diseases (FIMM; http://sdmc.krdl.org.sg:8080/fimm/), and (3) the ImMunoGeneTics database (IMGT; http://www.ebi.ac.uk/imgt/).

The invention is a method for the use of a trainable system to predict the binding of nucleic acids with proteins. This mode of prediction is carried out similarly to the antigenic determinant prediction scheme outlined above. Training data for local interactions between nucleic acid molecules (DNA, or RNA) and proteins are developed from the nucleic acid-protein complex structural data of the Protein Data Bank (PDB; http://www.rcsb.org/pdb/) and summarized in the DNA-Protein Interaction Database (DNAPIDB; http://www.dpidb.belozersky.msu.ru/). The sites of interaction are analyzed as before and converted to a set of features in the learning machine. The trained system outputs a thresholded-score indicative of the local propensity for nucleic acid binding at each site along the query protein.

The invention is a method for predicting biochemical, signal transduction and gene regulatory circuit pathways in the cell, using information obtained from the use of various modes of the trainable system to predict small molecule-protein, protein-protein, and protein-nucleic acid interaction pairs. Proteins analyzed by the trainable system may be subdivided based on cell compartment. Protein-protein interactions have been experimentally demonstrated using proteins that would never interact due to compartmentalization within cells. Proteins can be divided into groups based on cellular compartmentalization for entry into the trainable system for analysis (e.g. endoplasmic reticulum and Golgi apparatus for glycosylation machinery; nuclear proteins for DNA repair factors). Pathways may also be subdivided by the location of various processes in the cell. Signal transduction pathways involve the binding of small molecules by cell surface receptors (e.g. epidermal growth factor receptor, large G-protein receptors), followed by transmission of a signal via a number of cytosolic factors, some of which shuttle in and out of the nucleus, (e.g. kinases, adaptor proteins) to transcription factors in the nucleus (e.g. fos and jun). Thus, one can limit the potential interactions that can be determined by the use of the invention by limiting the input query to proteins that would have the opportunity to interact in the cell.

The invention is a method for cell-map proteomics. Biochemical, signaling and gene regulatory path ways can be mapped for entire organisms. The entire genome of the Helicobacter pylori, which contains coding sequences for 486 proteins, has been sequenced and 1,039 protein-protein interactions have been mapped. Using this model organism, which performs all of the functions required for viability, one can map the interactions of genomes of similar organisms, such as Campylobacter jejuni, an enteric bacteria pathogen that causes common symptoms of food poisoning. A complete protein-protein interaction map for C. jejuni computed using the methods disclosed herein is available at http://www-bioeng.ucsd.edu/cjbean/. Analysis of the major constituent protein domains shows a high degree of similarity. These orthologous bacterial proteomes represent a model system for demonstrating the utility of the invention for performing proteome wide interaction mining. The accuracy of the proteome map will depend on the quality of the database as well as the level of similarity of the organisms to be analyzed. The higher the similarity and the greater the number of interactions defined, the greater the predictive value of the information in the database.

### EXAMPLE 1

Databases of known biomolecular interactions. Databases of protein interactions are available at multiple sites including the Database of Interacting Proteins (DIP) http://dip.doe-mbi.ucla.edu which currently contains 10933 entries, and the H. pylori database, http://pim.hybrigenics.com which contains 1273 interacting pairs between the 486 potential proteins of the organism. In the DIP database, each interaction pair contains fields representing accession codes for other pubic protein databases, protein name identification and references to experimental literature underlying the interacting residue ranges, and protein-protein complex dissociation constants. The protein interaction domain coverage within the DIP is diverse; at least 175 distinct domains are represented. The proteins are predominantly eukaryotic, with a majority of the proteins being from the yeast Saccharomyces cerevisiae. The information in the database is updated constantly by individuals studying protein-protein interactions, thus providing an increasing number of interactions that may be “taught” to the trainable system of the invention.

A summary of public domain databases containing data appropriate for training this invention are listed in the following table. The entries in this table represent only a small subset of currently-available databases, which continue to appear and grow in size.

TABLE 1
Databases Useful for Training Systems Described in this Invention

| Database | Type of | | |
|---|---|---|---|
| Name | Data | Size | URL |
| Database of | Assorted | 10,933 | http://dip.dombi.ucla.edu |
| Interacting | protein- | interactions | |
| Proteins (DIP) | protein | | |
| | interactions | | |
| Protein | Whole- | 1273 protein- | http://pim.hybrigenics.com |
| Interaction Map | proteome | protein | |
| (PIM) | protein- | Interactions | |
| | protein | | |
| | interactions | | |
| | for H.pylori | | |
| Biomolecular | Protein- | 5939 | http://www.bind.ca/ |
| Interaction | protein | interactions, | |
| Network DB | interactions, | 54 | |
| (BIND) | molecular | complexes, 7 | |
| | complexes | pathways | |
| | and | | |
| | pathways | | |
| MIPS | Genetic, | Total | http://mips.gsf.de/proj/yeast/CYGD/ |
| Saccharomyces | physical | statistics | db/index.html |
| cerevisiae- | interactions | unlisted | |
| Interaction | in yeast | | |
| Tables | | | |
| Functional | functional | 400 protein | http://sdmc.krdl.org.sg:8080/fimm/ |
| IMMunology | immunology, | antigens, 1200 | |
| Database | focusing on | peptides, 800 | |
| (FIMM) | MHC, | HLA | |
| | antigens, | sequences, 50 | |
| | and disease | diseases | |
| SYFPEITHI | | Total | http://syfpeithi.bmi- |
| | | statistics | heidelberg.com/Scripts/MHCServer.dll/Info.htm#head |
| | | unlisted | |
| Drosophila | Drosophila | Total | http://cmmg.biosci.wayne.edu/finlab/PIMdb.htm |
| Protein | protein | statistics | |
| Interaction Map | interactions | unlisted | |
| Database | | | |
| DNA-Protein | 3D | Total | http://www.dpidb.belozersky.msu.ru/ |
| Interaction | structures of | statistics | |
| Database | complexes | unlisted | |
| (DNAPIDB) | in which | | |
| | protein | | |
| | binds either | | |
| | DNA or | | |
| | RNA | | |

### EXAMPLE 2

Support vector machine (SVM) learning. The protein-protein interaction estimator can utilize the technique of “support vector” learning, an area of statistical learning theory subject to extensive recent research (Vapnic, 1995; Schökopf et al., 1 999). The trainable system algorithm is not a limiting aspect of the invention. The method described in this invention can be used in conjunction with any exemplar-based machine learning paradigm, including, for example, neural networks, classification and regression trees (CART), or Bayesian networks. While in principle any of these or other learning algorithms would work with this invention, it is believed that SVM represents the best machine learning method for this invention, for the following reasons:

1. SVM generates a representation of the nonlinear mapping from biopolymer sequence to protein fold space using relatively few adjustable model parameters.

2. Based on the principle of structural risk minimization, SVM provides a principled means to estimate generalization performance via an analytic upper bound on the generalization error.

3. SVM is characterized by fast training, which is essential for high-throughput screening of large biological databases

The trainable system can be trained to classify labeled empirical data points by constructing an optimal high-dimensional decision function that (1) maximizes the separations between classes and (2) minimizes the “structural risk”

R(α)=∫Q(z, α) d F(z), αεΛ

with respect to perameters α using an independently, identically distributed (i.i.d.) sample Z=(z₁, z₂, . . . zᵢ) generated by an (unknown), underlying probability distribution F, where Q is an indicator function, and Λ is a set of parameters. Sample points zᵢ=(xᵢ, yᵢ) comprise protein features xᵢε [symbol] ⁿ and their classifications yᵢε{−1, 1}. In practice, the learning task converges rapidly as a constrained quadratic program is solved. The resultant decision function h represents an hypothesis generator for interference on novel data points, mapping them onto the discrete set y, or h:x→y. This is a binary decision (+1 [symbol] interaction, −1 [symbol] no interaction).

### EXAMPLE 3

Feature representation. For each amino acid sequence of a protein-protein complex, feature vectors were assembled from encoded representations of tabulated residue properties (Ratner et al., 1996) including charge, hydrophobicity and surface tension for each residue in the sequence. This set of features is not a limiting aspect of the invention. Instead any set of physical, chemical or biological features corresponding in a discrete or spatially-averaged sense to each residue or nucleotide in a linear biopolymer sequence may be used to construct an example for training the system described in this invention. These features are then concatenated to create an interaction pair example. Negative examples (i.e. putative non-interacting pairs) were generated by randomly extracting individual proteins from the database and randomizing their amino acid sequence while preserving their chemical composition. This randomization technique is well established for statistical significance estimation in biological sequence analysis.

### EXAMPLE 4

Analysis of protein-protein interactions using the DIP database. DIP database samples were at random, and data were partitioned into training and testing sets, at approximately a 1:1 ratio. Feature vectors were constructed in this manner and were used as examples for training and testing the learning machine. Testing examples were not exposed to the system during SVM learning. The database is robust in the sense that it represents a compendium of protein interaction data collected from diverse experiments. At least 175 protein domains are represented. There is a negligible probability that the learning system will “learn its own input” on a narrow, highly self-similar set of data examples. This enhances the generalization potential of the trained support vector machine.

Software methods for parsing the DIP database, control of randomization and sampling of records and sequences, and feature vector creation were developed in Java. A new database was constructed by augmenting the original DIP records. Additional fields added included amino acid sequence data and associated residue features as described in Example 3.

Support Vector Machine learning was implemented using Joachims' SVM^{light} (Joachims, 1999), available online at http://www-ai.cs.uni-dortmund.de/SOFTWARE/SVM_LIGHT.eng.html.

Training and testing exemplar data files were developed using maximum allowed residue length as an input parameter to the data preparation software. This threshold length was used to selectively filter out certain protein interactions from consideration as means to explore possible residue length dependence of the generalization accuracy of the SVM. A different SVM was trained for each maximum residue length threshold case. Residue length thresholds of of 350, 500, 750, 1000 and ∞ in the numerical experiments were considered.

The performance of each SVM was evaluated using the inductive accuracy of on the previously unseen samples as a metric. “Inductive accuracy” is defined here as the percentage of correct protein interaction predictions in the test set, including positive and negative interaction examples.

The main result of the protein-protein interaction predictions are summarized in the system generalization accuracy summary in Table 2. “Inductive accuracy” is the percentage of correct protein interaction predictions on test data not previously seen by the system. Each row in the table corresponds to a fixed residue length threshold used to generate the training and testing examples. Data in the column marked “# Examples” indicate the total number of training and testing examples for each case. During data preparation, at the shortest residue length thresholds, the random sampling procedure ignores database records more frequently as the threshold test is violated; this results in greater disparity between the train/test data counts.

| | # Examples | Inductive |
|---|---|---|
| Residue Cutoff | (train, test) | Accuracy |
| 350 | (122, 172) | 51.33% |
| 500 | (448, 380) | 67.37% |
| 750 | (1020, 1094) | 65.63% |
| 1000 | (1616, 1648) | 68.63% |
| ∞ | (2218, 2240) | 70.40% |

The data demonstrate that as the volume of available training data increases, nearly out of three potential protein interactions are correctly estimated system. When all of the data are included, the inductive accuracy reaches 70.4%. Apparently, even though the marginal contribution to the total protein interaction density function is very slight when including the longest protein in the analysis, these additional data points assist the SVM with the description of the margin. This observation is consistent with the nature of SVMs as margin classifiers, where a few key data examples near the decision boundary are sufficient to specify the boundary between the classes.

### EXAMPLE 5

Analysis of protein-nucleic acid interactions. The invention can be used to predict the binding of nucleic acids with proteins. This mode of prediction is carried out by casting the numerical optimization procedure as a regression problem. A continuous value for binding affinity of DNA/RNA-protein complex can be learned. In this manner the same scheme for representing linear biopolymer sequences as features is used, and the training procedure involves “sliding” a window along the query sequence, each step outputting a numerical value that constitutes a predicted interaction value of the sequence within the window and the query ligand.

Training data for local interactions between nucleic acid molecules (DNA, or RNA) and proteins can be developed from the nucleic acid-protein complex structural data of the Protein Data Bank (PDB; http://www.rcsb.org/pdb/) and summarized in the DNA-Protein Interaction Database (DNAPIDB; http://www.dpidb.belozersky.msu.ru/). The sites of interaction are analyzed as before and converted to a set of features in the learning machine. The trained system outputs a thresholded-score indicative of the local propensity for nucleic acid binding at each site along the query protein.

### EXAMPLE 6

Prediction of protein epitopes. The invention is a method for the use of a learning machine to predict the presence of epitopes of interest, including functional domains and binding sites of proteins, and antigenic determinants. The learning algorithm in this application is cast as a regression similarly to the DNA/RNA-protein determinant prediction scheme outlined above. Example public-domain databases containing data appropriate for training the system in this mode are: (1) The Ligand Chemical Database for Enzyme Reactions (http://www.genome.ad.jp/dbget/ligand.html), (2) The Function Immunology Database of MHC molecules, antigens and diseases (FIMM; http://sdmc.krdl.org.sg:8080/fimm/), and (3) the ImMunoGeneTics database (IMGT; http://www.ebi.ac.uk/imgt/).

### EXAMPLE 7

Whole proteome interaction analysis. The invention may be applied to larger scale studies of protein-protein interactions in a proteome wide scale. Application of a “phylogenetic bootstrap” method for protein-protein interaction mining, which comprises traversal of a phenogram, interleaving rounds of computation and experiment, to develop a knowledge base of protein interactions in genetically similar organisms. The steps comprising the phylogenetic bootstrap are distilled into an algorithm, described herein in detail.

The algorithm.

Input: Proteome sequences sₐ, s_{b}, labels Yₐ.

Input: Parameters δ, _{ε}_{_{cv}}^{max}

Assume: similarity ρ (F(Zₐ), F(Z_{b}))≦δ

Compute: feature set Xₐ, sample Zₐ

1. Xₐ [symbol] get Features (sₐ)

2. Zₐ⁺ [symbol] {(x, y)|x⊂Xₐ, y⊂Yₐ, y=+1}

3. Zₐ⁻ [symbol] {(x, y)|x⊂Xₐ, y⊂Yₐ, y=−1}

4. Zₐ [symbol] Zₐ^{+∪Z}ₐ⁻

Compute: decision rule on sample

5. h(α,x) [symbol] SVM (Zₐ)

Compute: C.V. generalization error estimate

6. _{ε}_{_{cv}} [symbol] L00({h})

7. Prob{ŷ=y|h}≈1−_{ε}_{_{cv}}

Assert: _{ε}_{_{cv}}^{v}≦_{ε}_{cv}?

Compute: feature set X_{b}

8. X_{b} [symbol] get Features (s_{b})

Compute: predict interactions

9. Ŷ_{b} [symbol] h (α, X_{b})

Assert: validate sample experimentally

10. Z_{b} [symbol] {(X,ŷ)|X⊂X_{b}, ŷ⊂ŷ

Assert: _{ε}_{_{cv}}^{v}≦_{ε}_{_{cv}}?

Input: New proteome sequences s_{c}

Update: sₐ, s_{b}, labels Yₐ

11. sₐ [symbol] sₐ+s_{b}; Yₐ [symbol] Yₐ+Ŷ_{b}; s_{b} [symbol] s_{c}

Goto: Step 1; iterate while _{ε}_{_{cv}}^{v}≦_{ε}_{_{cv}}≦_{ε}_{_{cv}}^{max}

The phylogenetic bootstrap algorithm above is summarized in this section. A procedural step identified by the pattern “S[num]” refers to Step #[num] in the accompanying Box entitled “Phylogenetic bootstrap algorithm”.

Input: First, it is necessary to specify the species Sₐ, S_{b} subject to investigation. In general, some existing protein interaction data may be at hand for each proteome, although their relative cardinality may be quite skewed, as discussed above. Our line of thought assumes that no interaction data are available for S_{b}; we have only a set of labels {Yₐ} corresponding to experimentally-verified interactions sampled from the proteome of species Sₐ, These labels, along with the amino acid sequence sets {sₐ} and {s_{b}} comprising the species' respective proteomes, are inputs to the algorithm.

Other inputs required are the inter-proteome distance δ (Eq. 2), and the maximum allowable rate of generalization error, ε_{cv}^{max}, where 0≦ε_{cv}^{max<}0.5.

S1-S4: Construct features based on attributes of the primary structure sequences {sₐ} from the training dataset. Encoded attributes Xₐ for entire proteomes may be derived from tabulated residue properties including charge, hydrophobicity, and surface tension as described previously (Bock and Gough, 2001). At this stage, data preprocessing including normalization and filtering should be performed to produce a useful sampled attribute set {x|x ε [symbol] ⁿ, ⊂X}. A total of / data points z are constructed by adding labels y to the accepted feature vectors x, or zᵢ=(xᵢ, yᵢ), i=1, . . . , l. The union of positively- and negatively-labeled examples constitutes the training sample {Zₐ}.

S5: Design an optimal support vector machine to classify data points in the sample {Zₐ}. After learning, the system builds a decision rule h that maps data vectors xᵢ onto the classification space yᵢε[−1,1]. The numerical sign of yᵢ is interpreted as the likelihood that the two proteins represented by xᵢ will interact.

S6-S7: Perform leave-one-out cross-validation experiments on the training set. For each observation zᵢ, train an SVM using all other points {z|zεZₐ, z≠zᵢ}., and predict the class membership of the omitted point zᵢ. Accumulate the total number of misclassifications observed in this process. Take the final average cross-validation error as the estimated generalization error rate ε_{cv} of the learner h.

S8: Construct features X_{b} from sequences {s_{b}} for the unlabeled proteome S_{b}. All-vs-all pairwise interactions may be represented in the prediction set. The same data preparation process should be applied as in S1.

S9: Predict a new set of protein-protein interactions {Ŷ_{b}} via the trained system; h(α): x_{b} [symbol] Ŷ_{b}, where α are parameters of the model. To the extent that the assumption of proteomic similarity ρ (F(Zₐ), F(Z_{b}))≦δ is satisfied, each point estimate ŷ is expected to be accurate with a probability g(δ)(1−ε_{cv}) or Prob {ŷ=y|h≈g(δ)(1−ε_{cv}).

S10: Take a random sample from the protein interaction prediction set Z_{b}={(x, ŷ)|x⊂X_{b}, ŷ⊂Y_{b}} and verify the predicted protein interactions (both positive and negative) using experimental proteomics techniques. Compare the experimentally-validated and calculated estimated prediction error rates. Assert that the following statement holds true: where the ε_{cv}^{v}≦ε_{cv}≦ε_{cv}^{max} superscript “v” denotes validate by experiment.

Input: Select sequences {s_{c}} from a new, related organism S_{c}. The similarity assumption ρ (F(Zₐ), F(Z_{c}))≦δ must still be maintained.

S11: Add sequences from the validated prediction set to the training set, and consider this expanded set as the training set for the next iteration: {sₐ}={sₐ}+{s_{b}}. Update the class labels by adding the prediction label set {Yₐ}={Yₐ}+{Ŷ_{b}}. Protein interactions for organism Sc will now be computed.

Return to S1 and repeat the process.

The stopping condition for this iteration is violation at any time of the assertions regarding the generalization error rate, i.e. when the error rate from LOO, ε_{cv} exceeds the specified limit ε_{cv}^{max}, or when the experimental observations contain more frequent errors than the calculated rate, or ε_{cv}^{v}>ε_{cv}.

Assumptions

The support vector machine (Vapnik, 2000) can be trained to classify labeled empirical data points by constructing an optimal high-dimensional decision function that (1) maximizes the separation between classes and (2) the minimizes “structural risk”

R(α)=∫Q(z, α) d F(z), αεΛ (1)

with respect to parameters using an independently, identically-distributed (i.i.d) sample Z=(z₁, z₂, . . . zᵢ) generated by an (unknown) underlying probability distribution F, where Q is an indicator function, and Λ is a set of parameters. Sample points zᵢ=(xᵢ, yᵢ) comprise protein features xᵢε [symbol] ⁿ and their classifications yᵢε{−1, 1}. In practice, the learning task converges rapidly as a constrained quadratic programming is solved. The resultant decision function h represents an hypothesis generator for inference on novel data points, mapping them onto the discrete set y, or h:x→y. This is a binary decision (+1 interaction, −1 no interaction). The assumption of a fixed generative probability distribution F(Z) in Eq. 1 is a key issue in the design of the data mining application. A consequence of this assumption is that a system trained on a sample Zₐ, taken from species Sₐ, may be used to predict interactions on a sample Z_{b} from another species S_{b}, provided that features of their respective protoeomes are not too dissimilar in some sense, or

ρ(F(Zₐ), F(Z_{b}))≦δ (2)

where is a distance metric and δ is a small positive constant. The statistic is general, and may signify cross-species similarity based on genome-level “edit distance” (Sanko. et al. 1992), whole-proteomic content (Tekaia et al. 1999), or molecular structures (Woese et al. 1990), to cite only three of many possibilities.

Interaction mining analysis as embodied in the phylogenetic bootstrap algorithm detailed above makes certain assumptions about the distributions of proteomic data in the design sample Z. Other assumptions inherent in this approach include:

1. Static intracellular state. If proteins A and B interact in species S1, they will also interact if co-occurring in species S2. This assumption may not be generally valid for different physiological conditions present in S2 relative to S1.

2. Completeness of design sample. Any pair of proteins (A,B) not labeled as interactors in the design sample Z are assumed to not interact. This is a subtle but significant point that must be held in mind when interpreting prediction results.

3. Proximity. The all-vs.-all computational screen selects interaction pairs based on primary structure, and does not discriminate protein subcellular location. Such analysis could be done in a separate post-mining filtering step.

4. Simple interactions. Only binary interactions are represented; complexes of proteins with more than two components are only inferred indirectly in post-mining analysis. This further implies that modifications to protein A (e.g., phosphorylation, glycosylation) prerequisite to its recognition by B are not identified.

Although an exemplary embodiment of the invention has been described above by way of example only, it will be understood by those skilled in the field that modifications may be made to the disclosed embodiment without departing from the scope of the invention, which is defined by the appended claims.

### REFERENCES

Champion, M. M. et al. (2001) Functional native-state proteomics in E. coli. In Proceedings of Proteomics: From Proteins to Drugs. San Francisco, Calif., Jun. 21-22, 2001. Cambridge Healthtech Institute.

Enright, A. J. et al. (1999) Protein interaction maps for complete genomes based on gene fusion events. Nature 402:86-90.

Fields, S. and O. -K. Song (1989) A novel genetic system to detect protein-protein interactions. Nature 340:245-6.

Joachims, T. (1999) Making Large-Scale Support Vector Machine Learning Practical. In Advances in Kernel Mehods—Support Vecotr Learning, ch. 11, pp. 169-84, MIT Press, Cambridge, Mass.

MacBeath, G. and S. L. Schreiber (2000) Putting proteins as microarrays for high throughput funciton determination. Science 289:1760-3.

Ratner, B. D. et al. (1996) Biomaterials Science: An Introduction to materials in Medicine, Academic Press, San Diego, Calif. 1996.

Sankoff, D. et al. (1992) Gene order comparisons of phylogenetic interference: Evolution of the mitochondrial genome. Proc. Natl. Acad, Sci. USA 89: 6575-9.

Schölkopf, B. et al. (1999) Advances in Kernel Methods: Support Vector Learning, MIT Press, Cambridge, Mass., 1999.

Tekia, F. et al. (1999) The genomic tree as revealed from a whole proteome comparisons. Genome Res. 9:550-7.

Vapnik, V. (1995) The Nature of Statistical Learning Theory. Springer-Verlag, New York, N.Y.

Woese, C. R. et al. (1990) Towards a natural system of organisms: Proposal for the domains Archea, Bacteria, and Eucarya. Proc. Natl. Acad. Sci. USA 87:4576-4579.

### We claim:

1. A method for predicting biomolecular interactions comprising,
inputting a training set comprising primary structure of biomolecules with known interactions into a trainable system,
inputting a set of biomolecules with unknown interactions into the trainable system, and
predicting interactions between members of the set of biomolecules with unknown interactions by analogy to the biomolecules in the training set using the trainable system.

2. The method of claim 1, wherein the interactions are homotypic.

3. The method of claim 1, wherein the interactions are heterotypic.

4. The method of claim 1, wherein the biomolecule is a protein.

5. The method of claim 1, wherein the biomolecule is a nucleic acid.

6. The method of claim 1, wherein the biomolecule is a bioactive agent.

7. A method for predicting whole proteome interactions comprising,
inputting a training set comprising all known protein-protein interactions from a single organism into a trainable system,
inputting a proteome of an organism with unknown interactions into the trainable system, and
predicting interactions between members of the set of proteins with unknown interactions using the trainable system.

8. A trainable system for predicting biomolecular interactions comprising,
a training set comprising primary structure of biomolecules with known interactions into a trainable system,
a set of biomolecules with unknown interactions into the trainable system, and
a system for predicting interactions between members of the set of biomolecules with unknown interactions by analogy to the biomolecules in the training set.
